(19) United States
(12) Patent Application Publication
(10) Pub. No.: US 2010/0222439 A1
(43) Pub. Date: Sep. 2, 2010

(54) BEVERAGE CONTAINING A POLYMERIC POLYPHENOL

(75) Inventors: Haitao Chen, Shanghai (CN); Weichang Liu, Shanghai (CN); Dabin Xie, Shanghai (CN)

Correspondence Address: UNILEVER PATENT GROUP, 800 SYLVAN AVENUE, AG West S. Wing, ENGLEWOOD CLIFFS, NJ, 07632-3100, US

(73) Assignee: CONOPCO, INC., D/B/A UNILEVER, Englewood Cliffs, NJ (US)

(21) Appl. No.: 12/711,294

(22) Filed: Feb. 24, 2010

(30) Foreign Application Priority Data
Feb. 27, 2009 (EP) EP09153957

(51) Int. Cl. A61K 31/05; A23F 3/00; A23F 3/20; A23L 2/78; A61K 8/34; A61P 43/00; A61Q 90/00

(52) U.S. Cl. 514/731; 426/597; 426/271

(57) ABSTRACT

This invention relates to a polymeric polyphenol containing beverage, In particular it relates to a substantially clear ambient temperature beverage comprising tea solids derived from fermented tea. The invention also relates to a method for improving the clarity of a polymeric polyphenol containing liquid composition.

It has long been observed that on cooling an aqueous black tea infusion from about 90 degrees Celsius to ambient temperature, a marked increase in the turbidity of the infusion can be seen leading ultimately to precipitation of up to about 30% w/w of the total tea solids. This precipitate is known as tea cream. It is thought that this precipitate originates from initial self-associations of polymeric polyphenols and association with caffeine thereby forming nano-clusters. These nano-clusters are not themselves responsible for any turbidity or precipitation of tea solids. However as the solubility of the polymeric polyphenols further reduces on cooling of the aqueous black tea infusion, these nano-clusters then aggregate into larger sub-micelles and ever larger micelles which are responsible for the turbidity and precipitate.

A solution to the aforementioned problem is provided in a first aspect of the invention by a beverage comprising tea solids, a liquid vehicle, added protein and added anionic polysaccharide,

wherein the beverage has a cream inhibition (CI) of 70-100%, preferably 80-100%, wherein CI=(1−((OD_{S}−OD_{B})/(OD_{O}−OD_{B})))100 where OD_{S} is the optical density of the beverage, OD_{B} is the optical density of the tea solids in a 25% w/w aqueous solution of ethanol and OD_{O} is the optical density of the beverage but in the absence of the protein and anionic polysaccharide additional to any in the tea solids, the optical density being measured at a fixed path length at 600 nm and at 4 degrees Celsius after equilibration at 4 degrees Celsius for 24 hours.

This invention relates to a beverage containing a polymeric polyphenol, in particular it relates to a substantially clear ambient temperature beverage comprising tea solids derived from fermented tea. The invention also relates to a method for improving the clarity of a polymeric polyphenol containing liquid composition.

It has long been observed that on cooling an aqueous black tea infusion from about 90 degrees Celsius to ambient temperature, a marked increase in the turbidity of the infusion can be seen leading ultimately to precipitation of up to about 30% w/w of the total tea solids. This precipitate is known as tea cream. It is thought that this precipitate originates from initial self-associations of polymeric polyphenols and association with caffeine thereby forming nano-clusters. These nano-clusters are not themselves responsible for any turbidity or precipitation of tea solids. However as the solubility of the polymeric polyphenols further reduces on cooling of the aqueous black tea infusion, these nano-clusters then aggregate into larger sub-micelles and ever larger micelles which are responsible for the turbidity and precipitate.

### SUMMARY OF THE INVENTION

A solution to the aforementioned problem is provided in a first aspect of the invention by a beverage comprising tea solids, a liquid vehicle, added protein and added anionic polysaccharide,

wherein the beverage has a cream inhibition (CI) of 70-100%, preferably 80-100%, wherein CI=(1−(OD_{S}−OD_{B})/(OD_{O}−OD_{B})))100 where OD_{S} is the optical density of the beverage, OD_{B} is the optical density of the tea solids in a 25% w/w aqueous solution of ethanol and OD_{O} is the optical density of the beverage but in the absence of the protein and anionic polysaccharide additional to any in the tea solids, the optical density being measured at a fixed path length at 600 nm and at 4 degrees Celsius after equilibration at 4 degrees Celsius for 24 hours.

By the term “beverage” is meant a substantially aqueous drinkable composition suitable for human consumption. Preferably the beverage comprises at least 85%, more preferably at least 90% and most preferably from 95 to 99.9% w/w water.

By the term “tea solids” is meant a dry material extractable from the leaves of the plant Camellia sinensis var. sinensis and/or Camellia sinensis var. assamica. The material will have been subjected to a so-called “fermentation” step wherein it is oxidised by certain endogenous enzymes that are released during the early stages of “black tea” manufacture. This oxidation may even be supplemented by the action of exogenous enzymes such as oxidases, laccases and peroxidases. Alternatively the material may have been partially fermented (“oolong” tea). In either case the tea solids will comprise polymeric polyphenols.

By the term “polymeric polyphenol” is meant compounds containing multiple hydroxyl groups attached to aromatic groups and having a molecular weight equal to or above 500 gram per mole. In the context of the present invention, the term polymeric polyphenol compound comprises oligomeric and polymeric polyphenol compounds. Preferably the molecular weight of the polymeric polyphenol compound is above 700 gram per mole, more preferred above 1000 gram per mole, most preferred above 1500 gram per mole.

The term ‘aromatic group’ includes aromatic hydrocarbon groups and/or heterocyclic aromatic groups. Heterocyclic aromatic groups include those containing oxygen, nitrogen, or sulphur (such as those groups derived from furan, pyrazole or thiazole). Aromatic groups can be monocyclic (for example as in benzene), bicyclic (for example as in naphthalene), or polycyclic (for example as in anthracene). Monocyclic aromatic groups include five-membered rings (such as those derived from pyrrole) or six-membered rings (such as those derived from pyridine). The aromatic groups may comprise fused aromatic groups comprising rings that share their connecting bonds. The term polyphenol also includes glycosidic polyphenols and/or their derivatives (e.g. acids, esters, and/or ethers). Any combinations of the free and various esterified, etherified and glycosylated forms of polyphenols are also included.

The polyphenol may be of natural origin (e.g. from tea, wine or chocolate), of synthetic origin, or mixtures thereof. With the term polymeric polyphenol compounds we include as examples for application in the present invention: tannic acid, condensed tannins, hydrolysable tannins, lignins, flavonoids, proanthocyanidins (or leucoanthocyanidins), procyanidins, theaflavins, thearubigins, theabrownins, tea haze, tea polyphenols (e.g. theasinensin, galloyl oolongtheanin, theaflavates and bistheaflavates), cocoa and wine polyphenols.

By the term “protein” is meant a polypeptide of weight average molecular weight 1000-10 000 000 Daltons.

By the term “polysaccharide” is meant a polymer of 40-3000 monosaccharide units.

By the terms “added protein” and “added anionic polysaccharide” are meant protein and anionic polysaccharide additional to that contained in the tea solids.

Polysaccharides and proteins are known to associate with each other through electrostatic interactions thereby to form a complex. Polysaccharides can hydrate and thereby enhance the solubility of the complex. As proteins are known to have a high affinity for polymeric polyphenols, the complex has both characteristics, ie high affinity for polymeric polyphenols and high solubility in water. Thus it is believed that the complex reduces the turbidity of cold aqueous black tea infusions by stabilising the polymeric polyphenol nano-clusters thereby preventing their aggregation into larger sub-micelles and ever larger micelles, or breaking up the sub-micelles or micelles back to nano-clusters. It has been found that a CI of at least 70% is required for a clear beverage.

A further advantage of the invention is that clear liquid compositions and in particular beverages may be produced with enhanced levels of polymeric polyphenol.

It has been observed that when a cationic polysaccharide, such as chitosan, is used, the beverage remains turbid. This is thought to be due to the weaker association between the positively charged polysaccharide and the now positively charged protein at low pH. Thus an anionic polysaccharide is essential in acid media.

Preferably the charge density of the added anionic polysaccharide is 0.25-20.00, preferably at least 0.30-20.00, most preferably at least 0.50-20.00 mole negative charge per mole of monosaccharide. It is thought that a higher charge density leads to a stronger association with the protein molecule and hence the complex formed is more robust. The added anionic polysaccharide may be selected from the group consisting of iota carrageenan, kappa carrageenan, lambda carrageenan, pectin, gum Arabic, propylene glycol alginate, alginate, cellulose and starch derivatives. Examples of cellulose and starch derivatives include carboxymethyl cellulose and phosphate starch respectively.

It has been observed that the beverage has improved clarity when the added protein has a tertiary structure and thus such added proteins are preferred. By the term “tertiary structure” is meant the stable structure defined by the spatial arrangement of secondary structures such as alpha helices and beta pleated sheets. Preferably the added protein is selected from the group consisting of caseinate, chicken egg white, bovine serum albumin and whey protein isolate. Whilst caseinate has relatively little tertiary structure or indeed secondary structure (the structure derived from stabilising repeating local structures with hydrogen bonds examples of which are the alpha helix and the beta pleated sheet), bovine serum albumin, whey protein isolate (a mixture of milk proteins) and chicken egg white all have tertiary structures.

The beverage may have a pH of 2.5 to 6.0, preferably 3.5 to 5.0. It is believed that at low pH, the association between polymeric polyphenols strengthens and makes tea cream, once formed, hard to dissolve so this invention is particularly useful in providing a clear low pH beverage comprising tea solids.

The weight ratio of added anionic polysaccharide to protein may be 20:1 to 1:4, preferably 15:1 to 1:2, most preferably 10:1 to 1:1. If too much protein is added, then the beverage will become more turbid as there is insufficient anionic polysaccharide to complex with the polymeric polyphenols. The weight ratio of the combination of added anionic polysaccharide and added protein to tea solids may be 0.001:1 to 1.0:1.0, preferably 0.001:1 to 0.5:1.0, most preferably 0.001:1 to 0.2:1.0. Limiting the amount of added anionic polysaccharide ensures that that the health benefits of tea are not outweighed by the negative impact of high levels of anionic polysaccharide.

In a second aspect of the invention, a method of improving the clarity of a liquid composition comprising a polymeric polyphenol is provided, the method comprising either sequentially in any order or simultaneously the steps of:

(a) adding a protein; and
(b) adding an anionic polysaccharide.

Performing step (b) either concurrent with or preceding step (a) has been observed to further improve clarity and is a preferred embodiment of the inventive method.

The protein and anionic polysaccharide may be selected from those set forth hereinabove for the added protein and the added anionic polysaccharide of the first aspect of the invention. The weight ratio of anionic polysaccharide to protein may be selected from those ratios also set forth hereinabove for the added protein and the added anionic polysaccharide of the first aspect of the invention.

The liquid composition may be a pharmaceutical product or a cosmetic product or a beverage, preferably a tea-based beverage.

By the term “tea-based beverage” is meant a beverage comprising at least 0.01%, preferably from 0.04-3%, more preferably from 0.06-2%, most preferably from 0.1-1% w/w tea solids.

### SUMMARY OF THE FIGURES

The invention is illustrated below with reference to:

FIGS. 1a and 1b which show the CI % for 5 mg/mL aqueous solutions of powdered tea with 0.001-1.25 mg/mL whey protein isolate (WPI) and 0.01-1.25 mg/mL iota carrageenan (IC) and the data of FIG. 1a configured to show the synergistic effect (CI %) respectively;

FIGS. 2a and 2b which show the CI % for 5 mg/mL aqueous solutions of powdered tea with 0.001-1.25 mg/mL whey protein isolate (WPI) and 0.01-1.25 mg/mL chitosan (a cationic polysaccharide) and the data of FIG. 2a configured to show the synergistic effect (CI %) respectively;

FIGS. 3a and 3b which show the CI % for 5 mg/mL aqueous solutions of powdered tea with 0.001-1.25 mg/mL whey protein isolate (WPI) and 0.01-1.25 mg/mL phosphate starch and the data of FIG. 3a configured to show the synergistic effect (CI %) respectively;

FIG. 4 which shows the optical density of a 5 mg/mL aqueous solution of powdered tea with 0.313 mg/mL whey protein isolate and 0.313 mg/mL iota carrageenan (see “0.5% tea infusion (+)”) compared to a sample prepared in the form of a 5 mg/mL aqueous powdered tea solution with 25% w/w ethanol (see “baseline”) and a 5 mg/mL aqueous solution of powdered tea (see “0.5% tea infusion (−)”) (without 0.313 mg/mL whey protein isolate and 0.313 mg/mL iota carrageenan) over 30 days at 4 degrees Celsius;

FIG. 5 which shows the gravimetric measurements of tea cream as % w/w tea cream for the 5 mg/mL aqueous solution of powdered tea with 0.313 mg/mL whey protein isolate and 0.313 mg/mL iota carrageenan (see “0.5% tea infusion (+)”) and the 5 mg/mL aqueous solution of powdered tea (see “0.5% tea infusion (−)”) over 10 days at 4 degrees Celsius;

FIG. 6 which shows the CI % for the powdered tea feeding model for procedure “A” and procedure “B”;

FIG. 7 which shows the optical density of aqueous solutions comprising theaflavins (TF4) on addition of a 1:1 by weight mixture of whey protein isolate and iota carrageenan (PP complex);

FIG. 8 which shows the optical density results of the theaflavins feeding model for procedure “A” and procedure “B”;

FIGS. 9a and 9b which show the CI % for 5 mg/mL aqueous solutions of powdered tea with 0.001-1.25 mg/mL bovine serum albumin (BSA) and 0.01-1.25 mg/mL sodium alginate and the data of FIG. 9a configured to show the synergistic effect (CI %) respectively;

FIGS. 10a and 10b which show the CI % for 5 mg/ml aqueous solutions of powdered tea with 0.001-1.25 mg/mL bovine serum albumin (BSA) and 0.01-1.25 mg/mL propylene glycol alginate (PGA) and the data of FIG. 10a configured to show the synergistic effect (CI %) respectively;

FIGS. 11a and 11b which show the CI % for 5 mg/mL aqueous solutions of powdered tea with 0.001-1.25 mg/mL bovine serum albumin (BSA) and 0.01-1.25 mg/mL kappa carrageenan (KC) and the data of FIG. 11a configured to show the synergistic effect (CI %) respectively;

FIGS. 12a and 12b which show the CI % for 5 mg/mL aqueous solutions of powdered tea with 0.001-1.25 mg/mL bovine serum albumin (BSA) and 0.01-1.25 mg/mL iota carrageenan (IC) and the data of FIG. 12a configured to show the synergistic effect (CI %) respectively;

FIGS. 13a and 13b which show the CI % for 5 mg/ml aqueous solutions of powdered tea with 0.001-1.25 mg/mL bovine serum albumin (BSA) and 0.01-1.25 mg/mL lambda carrageenan (IC) and the data of FIG. 13a configured to show the synergistic effect (CI %) respectively;

FIGS. 14a and 14b which show the CI % for 5 mg/mL aqueous solutions of powdered tea with 0.001-1.25 mg/mL bovine serum albumin (BSA) and 0.01-1.25 mg/mL gum Arabic and the data of FIG. 14a configured to show the synergistic effect (CI %) respectively;

FIGS. 15a and 15b which show the CI % for 5 mg/ml aqueous solutions of powdered tea with 0.001-1.25 mg/mL sodium caseinate and 0.01-1.25 mg/mL sodium alginate and the data of FIG. 15a configured to show the synergistic effect (CI %) respectively;

FIGS. 16a and 16b which show the CI % for 5 mg/mL aqueous solutions of powdered tea with 0.001-1.25 mg/mL sodium caseinate and 0.01-1.25 mg/mL propylene glycol alginate (PGA) and the data of FIG. 16a configured to show the synergistic effect (CI %) respectively;

FIGS. 17a and 17b which show the CI % for 5 mg/mL aqueous solutions of powdered tea with 0.001-1.25 mg/mL sodium caseinate and 0.01-1.25 mg/mL kappa carrageenan (KC) and the data of FIG. 17a configured to show the synergistic effect (CI %) respectively;

FIGS. 18a and 18b which show the CI % for 5 mg/mL aqueous solutions of powdered tea with 0.001-1.25 mg/mL sodium caseinate and 0.01-1.25 mg/mL iota carrageenan (IC) and the data of FIG. 18a configured to show the synergistic effect (CI %) respectively;

FIGS. 19a and 19b which show the CI % for 5 mg/mL aqueous solutions of powdered tea with 0.001-1.25 mg/mL sodium caseinate and 0.01-1.25 mg/mL lambda carrageenan (LC) and the data of FIG. 19a configured to show the synergistic effect (CI %) respectively; and

FIGS. 20a and 20b which show the CI % for 5 mg/mL aqueous solutions of powdered tea with 0.001-1.25 mg/mL sodium caseinate and 0.01-1.25 mg/mL gum Arabic and the data of FIG. 20a configured to show the synergistic effect (CI %) respectively

### DETAILED DESCRIPTION OF THE INVENTION

### Sample Preparation

5 mg/mL Aqueous Solution of Powdered Tea

A 5 mg/mL aqueous solution of Rupajuli Silvertippy freeze-dried powdered tea (Williamson Tea Assam Ltd.) was prepared by dissolving 0.5 g of powdered tea in 100 mL of 95° C. deionized water and centrifuging the mixture at 5,000 rpm for 5 minutes at 95° C. to remove any insoluble matter.

A 5 mg/mL Aqueous Solution of Powdered Tea with 0.313 mg/mL Whey Protein Isolate and 0.313 mg/mL Iota Carrageenan

A 5 mg/mL aqueous solution of Rupajuli Silvertippy freeze-dried powdered tea (Williamson Tea Assam Ltd.) with 0.313 mg/mL whey protein isolate (Alacen TM 895 from Fonterra Synergetic Group Ltd.) and 0.313 mg/mL iota carrageenan (Viscarin SD389 from FMC) was prepared by:
(a) Dissolving 1.0 g of powdered tea in 100 ml of 95° C. deionized water and centrifuging the mixture at 5,000 rpm for 5 minutes at 95° C. to remove any insoluble matter;
(b) Preparing a 1.25 mg/mL aqueous solution of whey protein isolate by dissolving 0.0625 g of whey protein isolate in 50 mL of deionized water at ambient temperature;
(c) Preparing a 1.25 mg/mL aqueous solution of iota carrageenan by dissolving 0.0625 g of iota carrageenan in 50 mL of deionized water at ambient temperature;
(d) Mixing the whey protein isolate solution and the iota carrageenan solution together at ambient temperature; and
(e) then combining the mixture of whey protein isolate and iota carrageenan held at ambient temperature with the powdered tea solution whilst held at 80 degrees Celsius.
   A 5 mg/mL Aqueous Solutions of Powdered Tea with 0.001-1.25 mg/mL Protein and 0.01-1.25 mg/mL Polysaccharide

Powdered tea solutions were prepared with a range of concentrations of protein and polysaccharide from 5 mg/mL stock solutions of protein and polysaccharide and a 10 mg/mL stock solution of powdered tea. The final concentrations of powdered tea, protein and polysaccharide were 5 mg/mL, 0.001-1.25 mg/mL and 0.01-1.25 mg/mL respectively. The solutions were prepared in a similar manner as for the 5 mg/mL aqueous solution of powdered tea with 0.313 mg/mL whey protein isolate and 0.313 mg/mL iota carrageenan hereinabove. The proteins used, apart from whey protein isolate were type A gelatine (G1890 from Sigma), bovine serum albumin (Sinopharm Chemical Reagent Co. Ltd.) and sodium caseinate (C8654 from Sigma). The polysaccharides used apart from iota carrageenan were sodium alginate, propylene glycol alginate, gum Arabic, kappa carrageenan, chitosan, phosphate starch and lambda carrageenan (22049 from Sigma). A blank sample was prepared in the form of a 5 mg/mL aqueous powdered tea solution with 25% w/w ethanol. The samples were prepared in a 96 well plate.

### Powdered Tea Feeding Model

A 10 mg/mL aqueous solution of powdered tea was prepared in the same manner as previously described. 1.25 mg/mL aqueous solutions of whey protein isolate and iota carrageenan were prepared and all three solutions combined to yield a solution with 5 mg/mL powdered tea, 0.313 mg/mL whey protein isolate and 0.313 mg/mL iota carrageenan. The following feeding programme was used with the 10 mg/mL aqueous solution of powdered tea being either at ambient temperature (reference “Procedure B”) or at 80 degrees Celsius (reference “Procedure A”) when combined with the ambient temperature 1.25 mg/mL aqueous solutions of whey protein isolate and iota carrageenan:

T+WPI/IC: 25 mL whey protein isolate solution and 25 mL iota carrageenan solution are premixed and added to 50 mL powdered tea solution.
T+IC: 25 mL iota carrageenan solution is added to 50 mL of powdered to solution and 25 mL of deionised water added.
T+WPI: 25 mL whey protein isolate solution is added to 50 mL of powdered tea solution and 25 mL of deionised water added.
TWPI+IC “x” min: 25 mL whey protein isolate solution is added to 50 mL powdered tea solution, and then 25 mL iota carrageenan solution is added after 5 or 10 or 30 minutes (where “x” is 5, 10 or 30).
TIC+WPI x min: 25 mL iota carrageenan solution is added to 50 mL powdered tea solution, and then 25 mL whey protein isolate solution is added after 5 or 10 or 30 minutes (where “x” is 5, 10 or 30).
Aqueous Solutions of 0.19-1.5 mg/mL Theaflavins and 0.078-2.5 mg/mL 1:1 by Weight Mixture of Whey Protein Isolate and Iota Carrageenan

A 5 mg/mL 1:1 by weight mixture of whey protein isolate and iota carrageenan aqueous solution was prepared. An aqueous solution of theaflavins (Theaflavin 4, which is a mixture of theaflavin, theaflavin 3-O-gallate, theaflavin 3′-O-gallate and theaflavin 3,3′-O-gallate with total theaflavins content of 95% w/w prepared in-house) was prepared at 80 degrees Celsius. The two solutions were combined maintaining the aqueous solution of theaflavins at 80 degrees Celsius to give solutions with a range of concentrations of the mixture and theaflavins.

### Theaflavins Feeding Model

A 3 mg/mL aqueous solution of theaflavins was prepared at 80 degrees Celsius. 0.625 mg/mL aqueous solutions of whey protein isolate and iota carrageenan were prepared and all three solutions combined to yield a solution with 1.5 mg/mL theaflavins, 0.156 mg/mL whey protein isolate and 0.156 mg/mL iota carrageenan. The following feeding programme was used with the 3 mg/mL aqueous solution of theaflavins being either at ambient temperature (reference “Procedure B”) or at 80 degrees Celsius (reference “Procedure A”) when combined with the ambient temperature 0.625 mg/mL aqueous solutions of whey protein isolate and iota carrageenan:

TF+WPI/IC: 25 mL whey protein isolate solution and 25 mL iota carrageenan solution are premixed and added to 50 mL theaflavins solution.
TF+IC: 25 mL iota carrageenan solution is added to 50 mL of theaflavins solution and 25 mL of deionised water added.
TF+WPI: 25 mL whey protein isolate solution is added to 50 mL of theaflavins solution and 25 mL of deionised water added.
TFWPI+IC: 25 mL whey protein isolate solution is added to 50 mL theaflavins solution, and then 25 mL iota carrageenan solution is added after 10 minutes.
TFIC+WPI: 25 mL iota carrageenan solution is added to 50 mL theaflavins solution, and then 25 mL whey protein isolate solution is added after 10 minutes.

### Cold Water Soluble Theaflavins Solutions

25 mL of 3 mg/mL aqueous solution of theaflavins and a 5 mg/mL aqueous solution of 1:1 by weight mixture of whey protein isolate and iota carrageenan were prepared at 80 degrees Celsius and at ambient temperature respectively. 3.125 mL of the 5 mg/mL aqueous solution of 1:1 by weight mixture of whey protein isolate and iota carrageenan was diluted to 25 ml with deionised water and mixed with the 25 mL 3 mg/mL aqueous solution of theaflavins whilst the latter was held at 80 degrees Celsius, yielding a 50 ml aqueous solution of 1.5 mg/ml theaflavins and 0.3125 mg/mL of 1:1 by weight mixture of whey protein isolate and iota carrageenan. The 50 mL solution was divided into two equal parts and one frozen at −40 degrees Celsius and the other frozen with liquid nitrogen. Each part was then freeze dried to produce a powder.

### Tests

### Measurement of Cream Inhibition

All the samples were stored at 4 degrees Celsius for 24 hours in order to equilibrate the tea creaming process before measurement of cream inhibition (CI). The optical density was measured at 600 nm and cream inhibition (CI %) calculated from the following equation:

$$CI \% = ( 1 - \frac{{OD}_{s} - {OD}_{B}}{{OD}_{O} - {OD}_{B}} ) \times 100 \%$$

where OD_{S} is the optical density of the beverage, OD_{B} is the optical density of the tea solids in a 25% w/w aqueous solution of ethanol and OD_{O} is the optical density of the beverage but in the absence of the protein and anionic polysaccharide additional to any in the tea solids, the optical density being measured at a fixed path length at 600 nm and at 4 degrees Celsius after equilibration at 4 degrees Celsius for 24 hours.

### Measurement of Turbidity

All the samples were stored at 4 degrees Celsius for 24 hours in order to equilibrate the tea creaming process before measurement of turbidity at 600 nm with a Safire2™ microplate reader (Tecan Group Ltd.).

### Gravimetric Measurement of Tea Cream

All the samples were stored at 4 degrees Celsius for 24 hours in order to equilibrate the tea creaming process before gravimetric measurement of tea cream. Then the samples were centrifuged at 10000 rpm for 10 minutes at 4 degrees Celsius, any sediment dried at 90 degrees Celsius for 24 hours and the dried sediment weighed.

### Results and Discussion

5 mg/mL Aqueous Solutions of Powdered Tea with 0.001-1.25 mg/mL Whey Protein Isolate and 0.01-1.25 mg/mL Polysaccharide

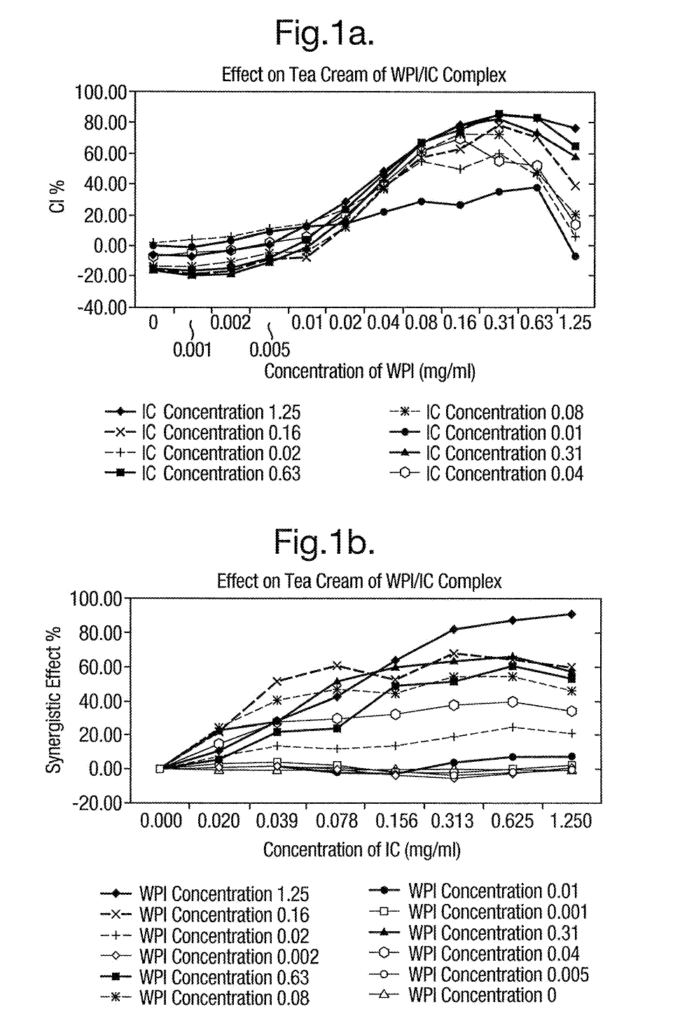

FIG. 1a shows the CI % for the solutions prepared hereinabove for whey protein isolate (WPI) and iota carrageenan (IC). The synergistic effect between the protein and polysaccharide is defined as CI % for the mixture of protein and polysaccharide (CI % pp) less the CI % for protein only (CI % pr) and the CI % for the polysaccharide only (CI % ps), thus Synergistic effect=CI %(pp)−CI % (pr)−CI %(ps). The recalculated results from FIG. 1a are shown in FIG. 1b which illustrates a clear synergistic effect with increasing protein concentration and increasing polysaccharide concentration.

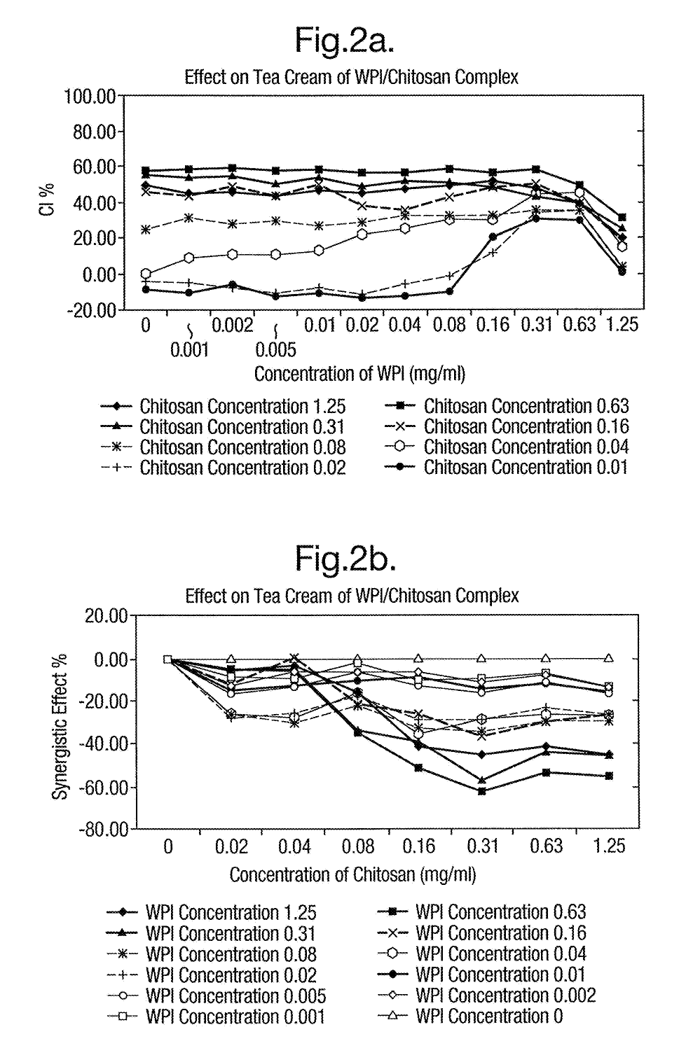

FIGS. 2a and 2b show the CI % for 5 mg/mL aqueous solutions of powdered tea with 0.001-1.25 mg/mL whey protein isolate (WPI) and 0.01-1.25 mg/mL chitosan (a cationic polysaccharide) and the data of FIG. 2a configured to show the synergistic effect (CI %) respectively. It can be seen that chitosan performs poorly with whey protein isolate and this is believed to be due to the fact that as chitosan can only be solubilised at low pH such as 3.0 (in this case at pH 3.0 with citric acid), the polysaccharide molecule becomes positively charged and thus less associated with the protein which is also positively charge.

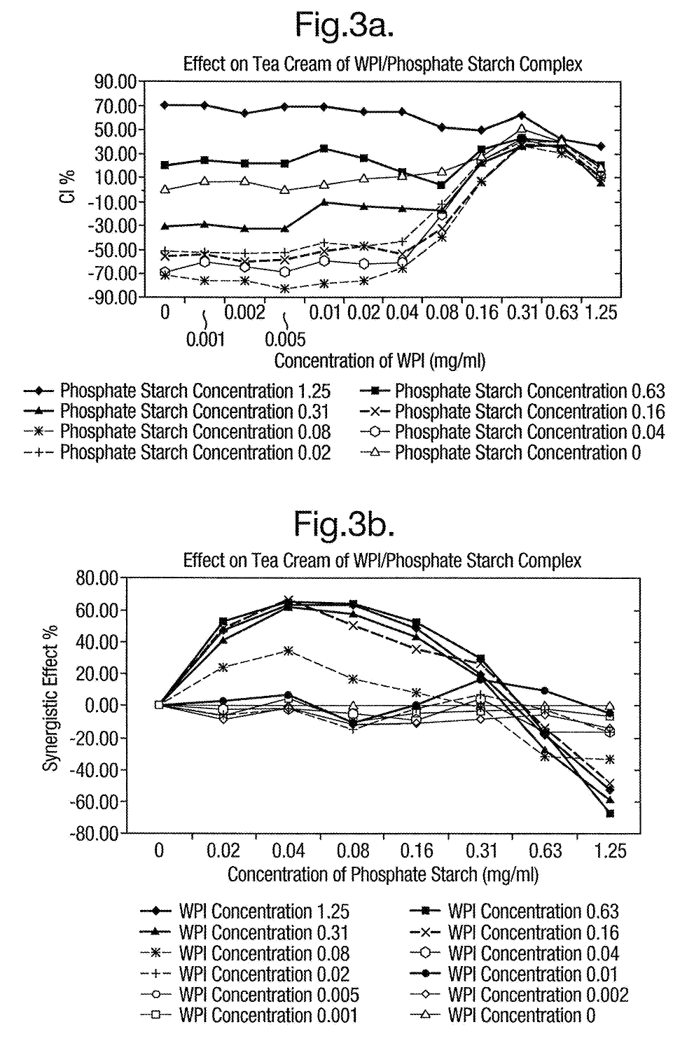

FIGS. 3a and 3b show the CI % for 5 mg/mL aqueous solutions of powdered tea with 0.001-1.25 mg/ml whey protein isolate (WPI) and 0.01-1.25 mg/mL phosphate starch and the data of FIG. 3a configured to show the synergistic effect (CI %) respectively.

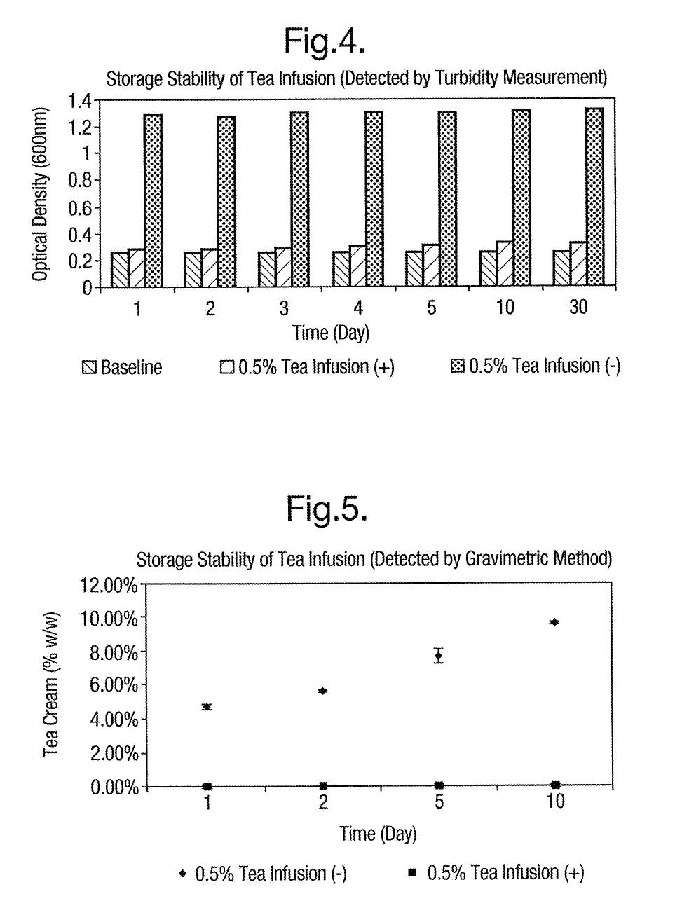

The ability of the whey protein isolate-iota carrageenan complex to stabilise the polymeric polyphenol nano-clusters is illustrated in FIG. 4 which shows that the optical density of a 5 mg/mL aqueous solution of powdered tea with 0.313 mg/mL whey protein isolate and 0.313 mg/mL iota carrageenan (see “0.5% tea infusion (+)”); the preparation of which has been previously described, over 30 days at 4 degrees Celsius hardly changes. The “baseline” and “0.5% tea infusion (−)” represent respectively a sample prepared in the form of a 5 mg/mL aqueous powdered tea solution with 25% w/w ethanol and the 5 mg/mL aqueous solution of powdered tea (without 0.313 mg/mL whey protein isolate and 0.313 mg/mL iota carrageenan) the preparation of which has been previously described. FIG. 5 shows the gravimetric measurements of cream tea as w/w (%) tea cream for the 5 mg/mL aqueous solution of powdered tea with 0.313 mg/mL whey protein isolate and 0.313 mg/mL iota carrageenan (see “0.5% tea infusion (+)”) and the 5 mg/mL aqueous solution of powdered tea (see “0.5% tea infusion (−)”) over 10 days at 4 degrees Celsius. The gravimetry results confirm the optical density results.

### Powdered Tea Feeding Model

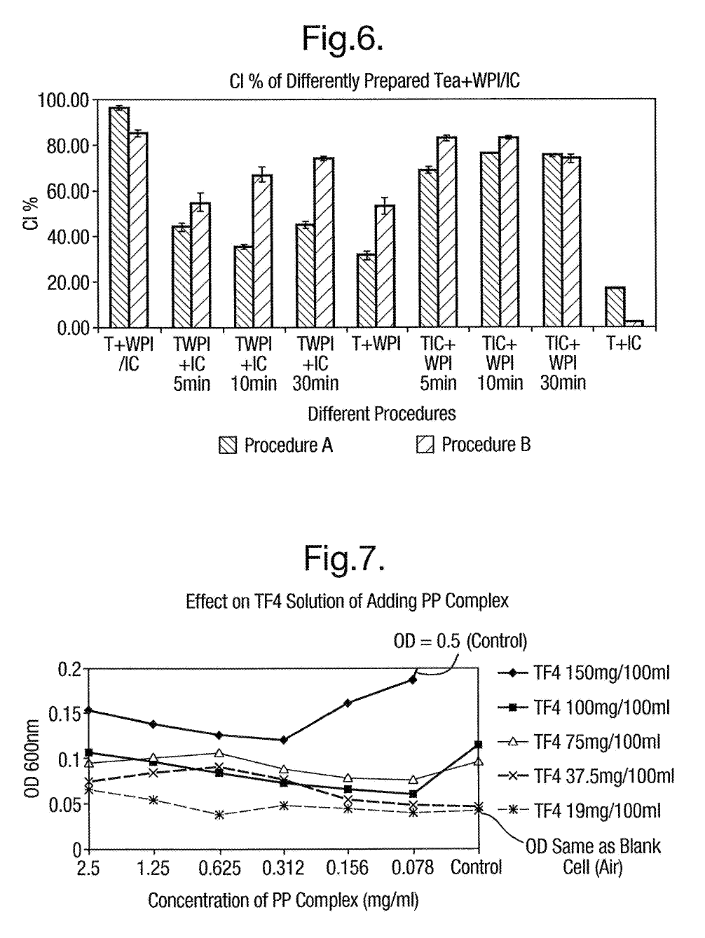

FIG. 6 shows the results of the powdered tea feeding model from which the following conclusions can be drawn:
1) Feeding model effect: The protein-polysaccharide complex has a higher CI % than adding protein and polysaccharide separately whatever the tea solution's temperature.
2) Temperature effect: Adding protein at 80° C. gives a lower CI % compared to adding protein at ambient temperature. This may be due to the denaturing of the protein or enhanced protein-polyphenol interaction at higher temperatures.
3) Polysaccharides effect: Adding the protein first gives a lower CI % compared to adding the polysaccharide first. However adding further polysaccharide increases CI %, implying the polysaccharide can increase the solubility or the dispersibility of the protein-polyphenol complex.
4) Proteins effect: Adding the polysaccharide alone does not significantly improve the CI % but adding the protein gives a comparably high CI % similar to adding the protein-polysaccharide complex. It might imply that the polysaccharide-polyphenol association is weaker than the protein-polyphenol association.
   Aqueous Solutions of 0.19-1.5 mg/mL Theaflavins and 0.078-2.5 mg/mL 1:1 by Weight Mixture of Whey Protein Isolate and Iota Carrageenan

FIG. 7 shows the positive effect on the clarity of aqueous theaflavins (TF4) solutions on addition of a 1:1 by weight mixture of whey protein isolate and iota carrageenan (PP complex).

### Theaflavins Feeding Model

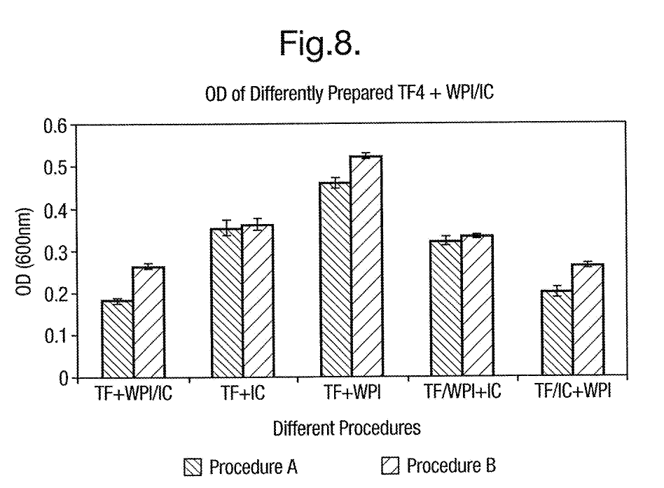

FIG. 8 shows the results of the theaflavins feeding model from which the following conclusions can be drawn:
1) Adding iota carrageenan alone decreases the solubility of theaflavin.
2) Adding whey protein isolate alone decreases the solubility of theaflavin and results in precipitation due to polymeric polyphenol-protein association.
3) Adding iota carrageenan first yields an optical density comparable to that of simulataneous addition of whey protein isolate and iota carrageenan.
4) Adding whey protein isolate first yields a lower optical density than that of whey protein isolate alone, indicating that iota carrageenan can increase the solubility of the theaflavins-whey protein isolate association.

### Cold Water Soluble Theaflavins Solutions

The freeze dried powders were redispersed in deionised water at final concentrations of 1.5, 4.5 and 9.0 mg/ml theaflavins and the results are tabulated hereinbelow.

TABLE 1
Appearance of aqueous solutions of theaflavins prepared as indicated.

| | | Freeze dried | |
|---|---|---|---|
| | | product of | Freeze dried product of |
| Final theaflavins | | theaflavins solution | theaflavins solution after |
| concentration | | after freezing in | freezing at −40 degrees |
| (mg/mL) | Theaflavins only | liquid N₂ | Celsius |
| 1.5 | Hard to dissolve, | Dissolves | Dissolves completely with |
| | some sediment, | completely with no | no sediment. |
| | need heat to | sediment. | |
| | solubilise | | |
| 4.5 | — | Requires stirring to | Requires stirring to |
| | | dissolve, stable at 4 | dissolve, stable at 4 |
| | | degrees Celsius | degrees Celsius |
| 9 | — | Requires stirring to | — |
| | | dissolve, not stable | |
| | | at 4 degrees Celsius | |

Freeze drying of a 1.5 mg/mL theaflavins and 0.3125 mg/mL of 1:1 by weight mixture of whey protein isolate and iota carrageenan which has been frozen in liquid N₂ or at −40 degrees Celsius yields a dry product which can be incorporated into deionised water at theaflavin levels up to three times more than untreated theaflavins. It is thought that freeze drying does not significantly disrupt the association between polysaccharide and protein resulting which would result in poorer performance at stabilizing the polymeric polyphenol nano-clusters before they aggregate into larger sub-micelles and ever larger micelles which are responsible for turbidity and precipitate.

5 mg/mL Aqueous Solutions of Powdered Tea with 0.001-1.25 mg/mL Bovine Serum Albumin and 0.01-1.25 mg/mL Polysaccharide

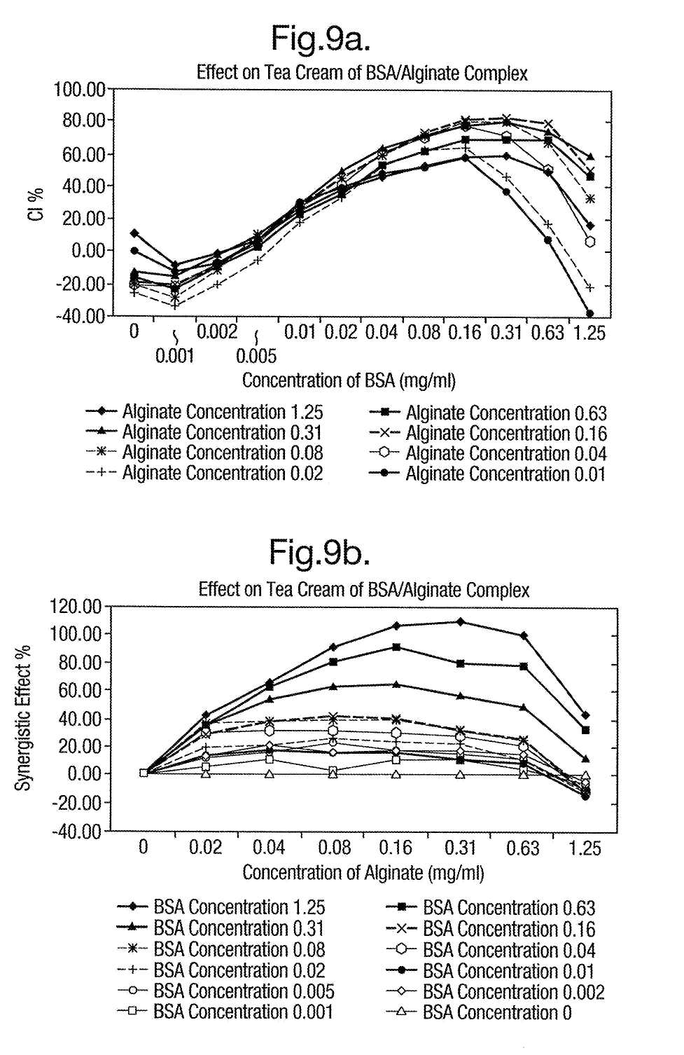

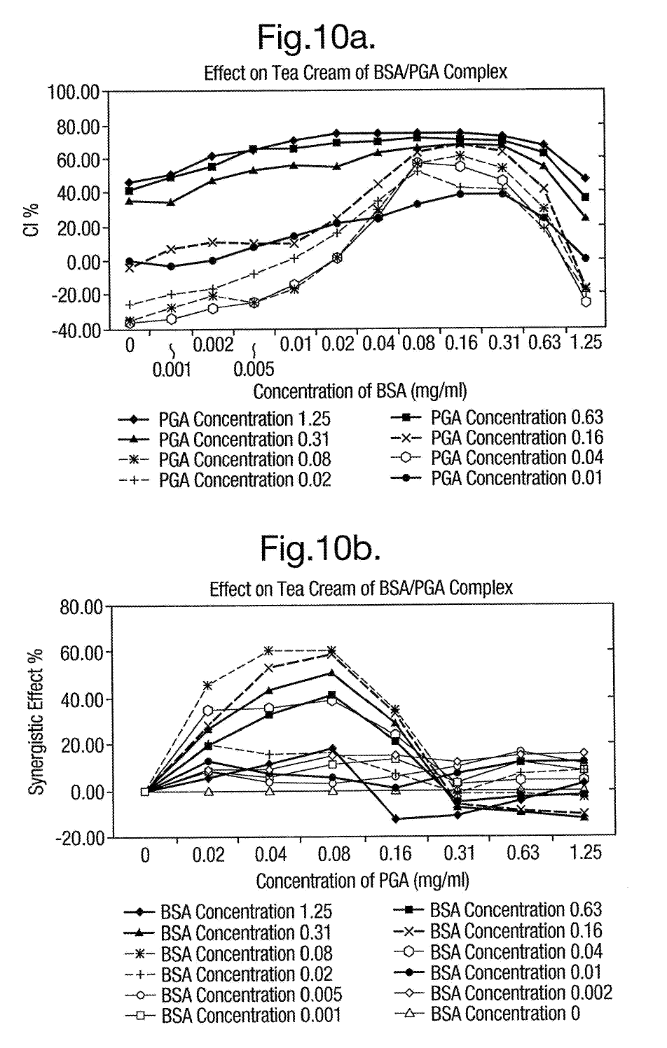

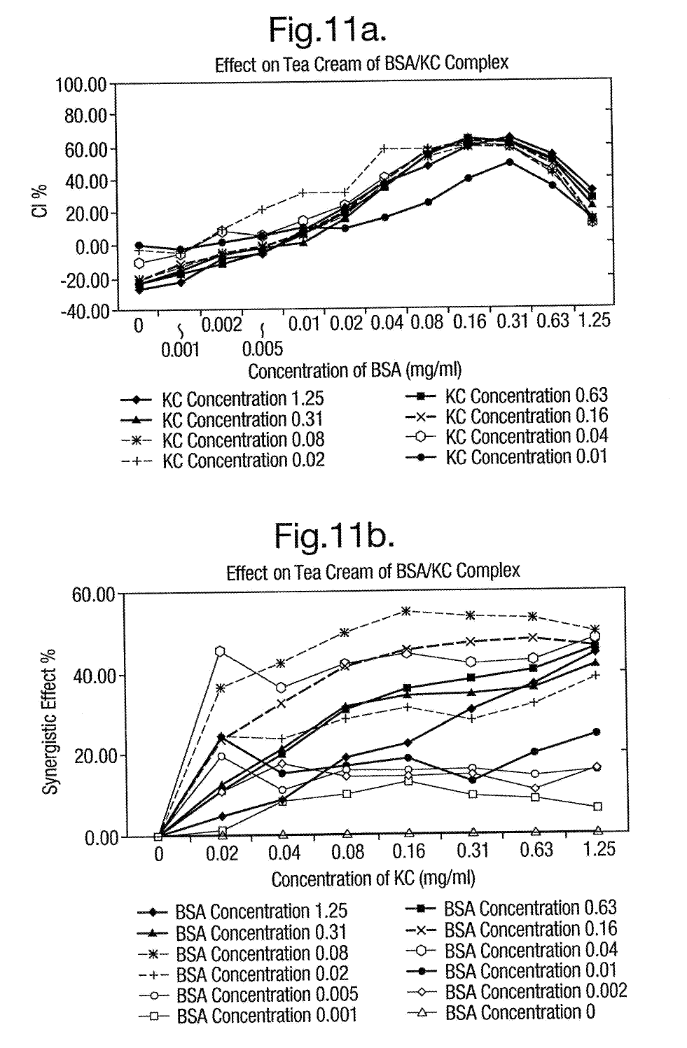

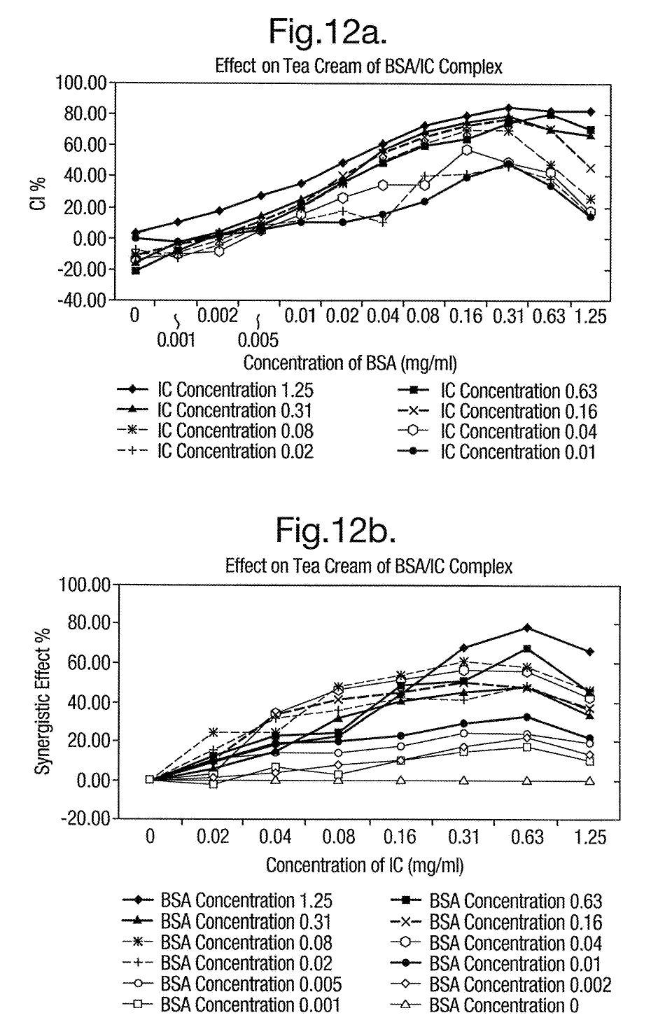

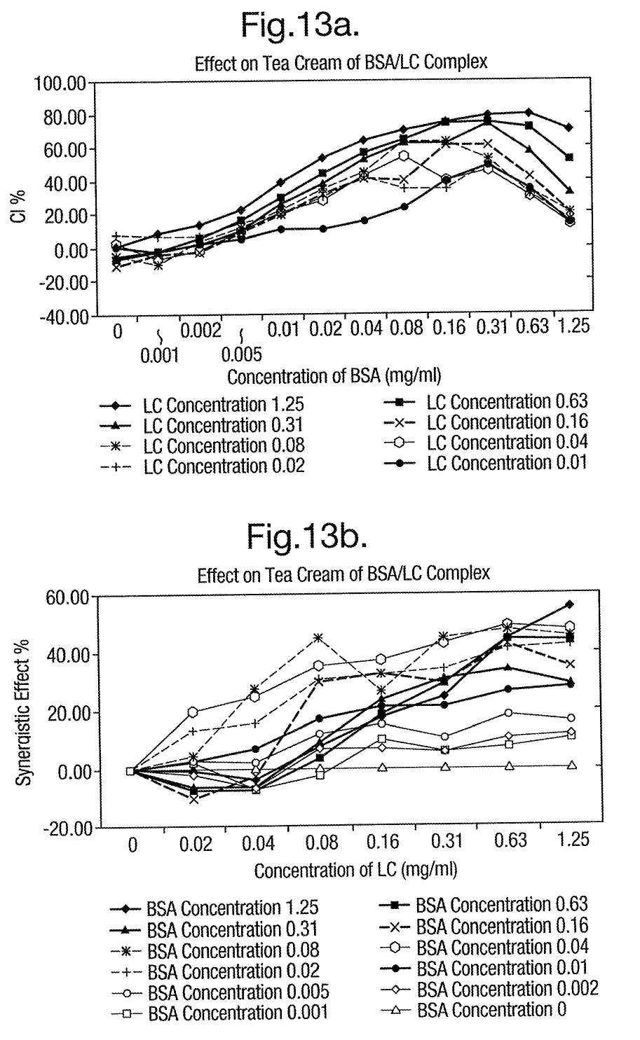

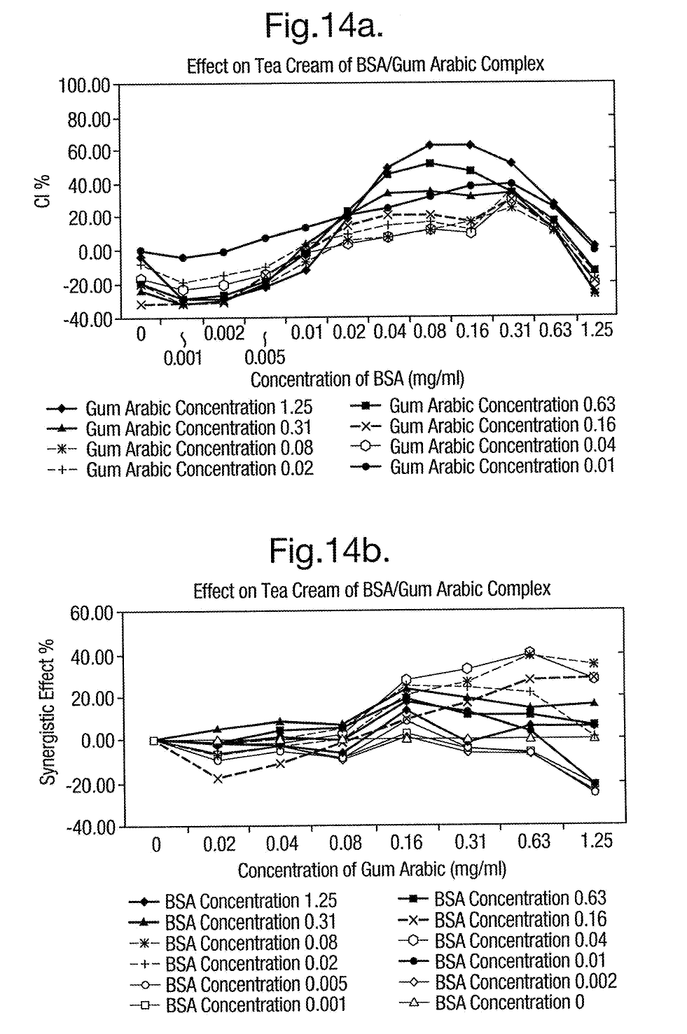

FIGS. 9a, 10a, 11a, 12a, 13a and 14a show the CI % for 5 mg/mL aqueous solutions of powdered tea and 0.001-1.25 mg/mL bovine serum albumin (BSA) with respectively 0.01-1.25 mg/mL sodium alginate, propylene glycol alginate (PGA), kappa carrageenan (KC), iota carrageenan (IC), lambda carrageenan (LC) and gum Arabic. FIGS. 9b, 10b, 11b, 12b, 13b and 14b respectively show the data of FIGS. 9a, 10a, 11a, 12a, 13a and 14a configured to show the synergistic effect (CI %).

The charge densities (negative charge per mole of monosaccharide) of the polysaccharides is:

| | |
|---|---|
| Sodium alginate | 1.0 |
| Propylene glycol alginate | <1.0 |
| Kappa carrageenan | 0.5 |
| Iota carrageenan | 1.0 |
| Lambda carrageenan | 1.5 |
| Gum Arabic | Low |

The synergistic effect can be ranked sodium alginate>>iota carrageenan>propylene glycol alginate>kappa carrageenan=lambda carrageenan>gum Arabic. Clear beverages are obtained with all the polysaccharides with the exception of gum Arabic because, it is believed, the charge density is too low. As propylene glycol alginate has a lower charge density, the synergistic effect between this polysaccharide and whey protein isolate is less pronounced than when using sodium alginate.

5 mg/mL Aqueous Solutions of Powdered Tea with 0.001-1.25 mg/mL Type A Gelatine and 0.01-1.25 mg/mL Polysaccharide

The CI % for 5 mg/mL aqueous solutions of powdered tea and 0.001-1.25 mg/mL type A gelatine with respectively 0.01-1.25 mg/mL sodium alginate, kappa carrageenan (KC), iota carrageenan (IC), lambda carrageenan (LC) and gum Arabic were determined and all found to be below 70%. Type A gelatine has little tertiary structure as it is derived from partial hydrolysis of collagen during which the intermolecular and intramolecular bonds which stabilise collagen are broken as well as the hydrogen bonds stabilising the collagen helix. Indeed type A gelatine has little secondary structure either.

5 mg/mL Aqueous Solutions of Powdered Tea with 0.001-1.25 mg/mL Sodium Caseinate and 0.01-1.25 mg/mL Polysaccharide

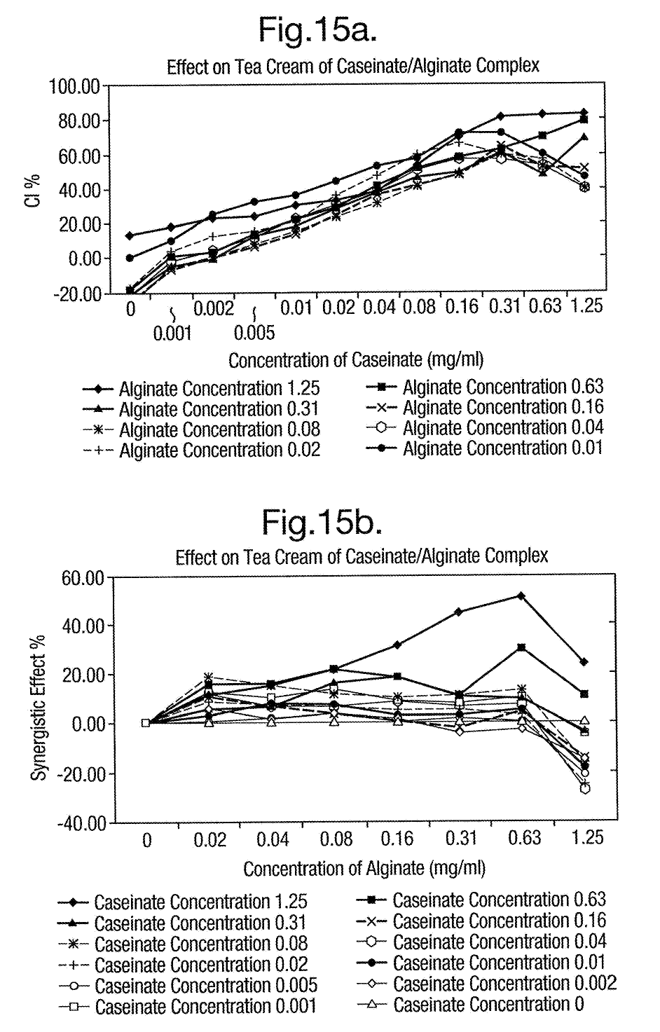

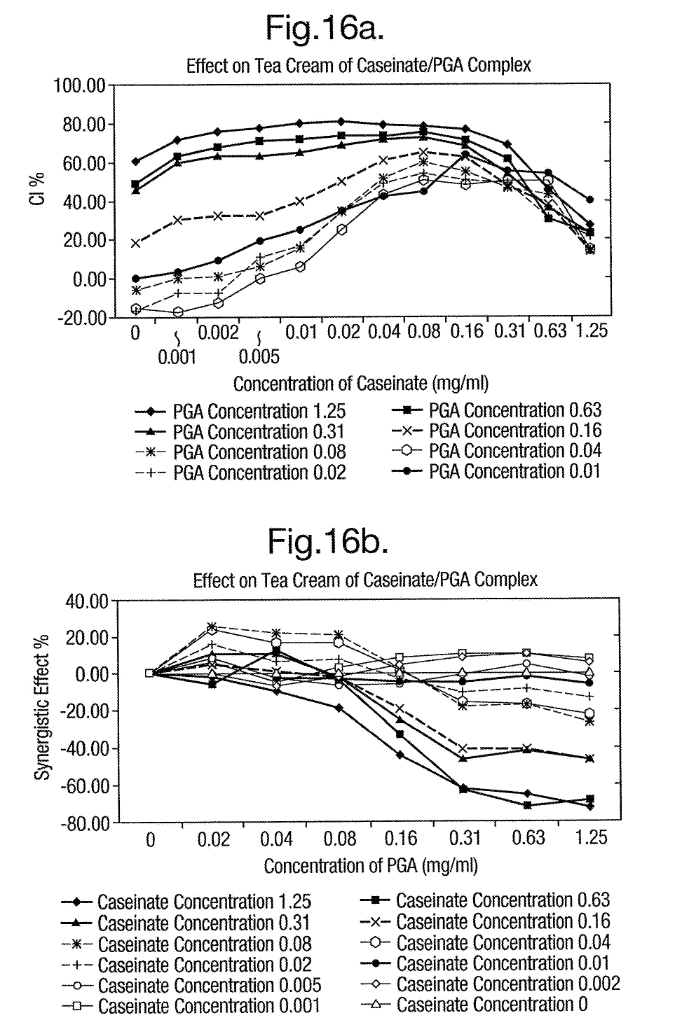

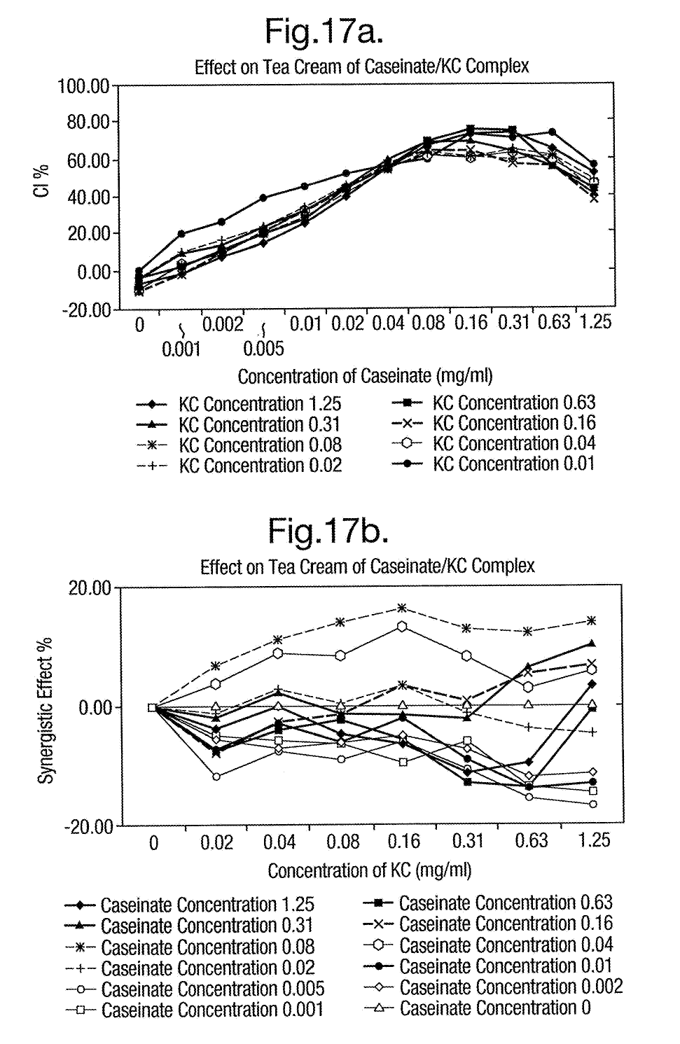

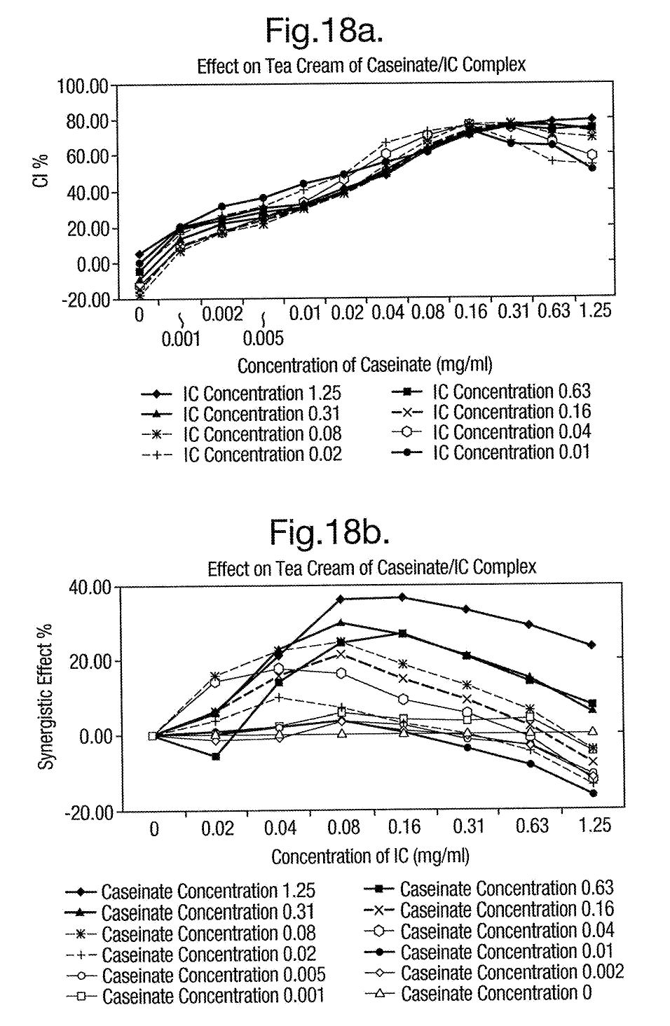

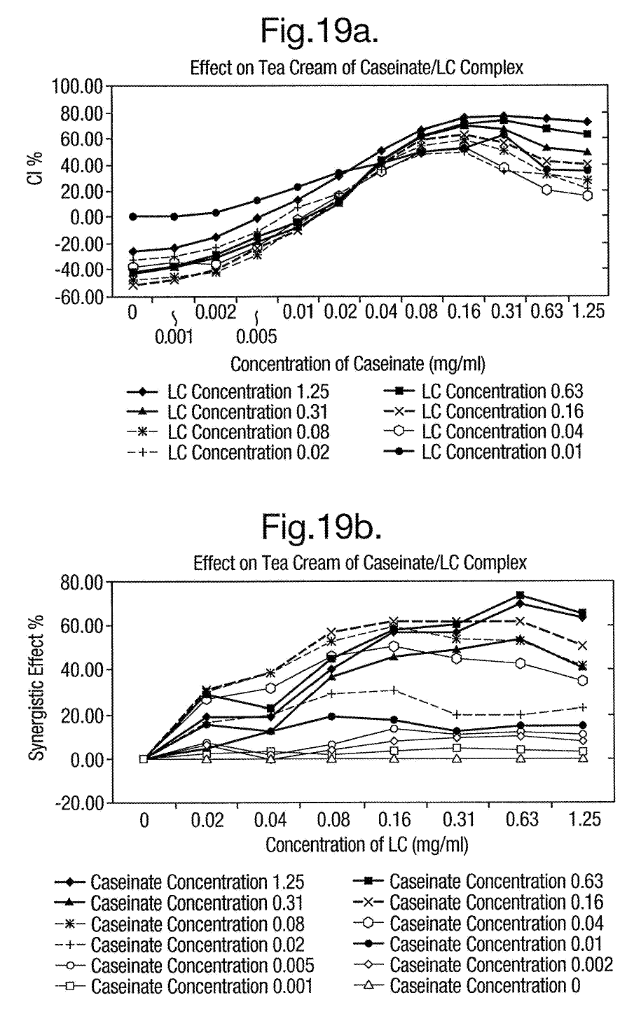

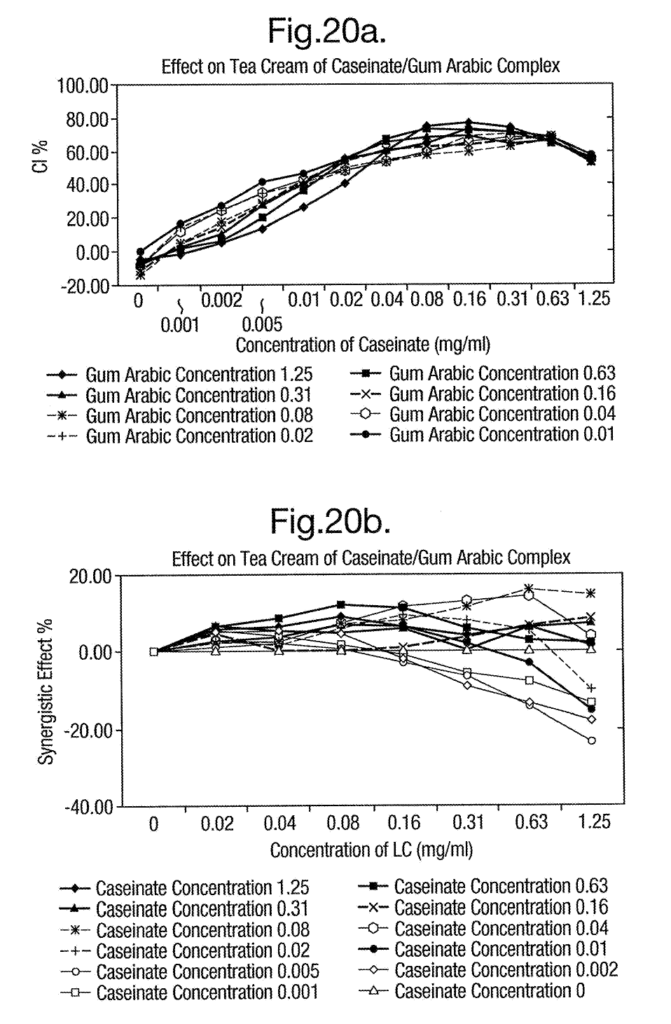

FIGS. 15a, 16a, 17a, 18a, 19a and 20a show the CI % for 5 mg/mL aqueous solutions of powdered tea and 0.001-1.25 mg/mL sodium caseinate with respectively 0.01-1.25 mg/mL sodium alginate, propylene glycol alginate (PGA), kappa carrageenan (KC), iota carrageenan (IC), lambda carrageenan (LC) and gum Arabic. FIGS. 15b, 16b, 17b, 18b, 19b and 20b respectively show the data of FIGS. 15a, 16a, 17a, 18a, 19a and 20a configured to show the synergistic effect (CI %).

Whilst clear beverages are produced, it is clear there is little synergy between the sodium caseinate and the polysaccharides. It is postulated that this may be due to the fact that sodium caseinate has little tertiary structure.

1. A beverage comprising tea solids, a liquid vehicle, added protein and added anionic polysaccharide,
wherein the beverage has a cream inhibition (CI) of 70-100%, preferably 80-100%, wherein CI=(1−((OD_{S}−OD_{B})/(OD_{O}−OD_{B})))100 where OD_{S} is the optical density of the beverage, OD_{B} is the optical density of the tea solids in a 25% w/w aqueous solution of ethanol and OD_{O} is the optical density of the beverage but in the absence of the protein and anionic polysaccharide additional to any in the tea solids, the optical density being measured at a fixed path length at 600 nm and at 4 degrees Celsius after equilibration at 4 degrees Celsius for 24 hours.

2. A beverage according to claim 1, wherein the charge density of the added anionic polysaccharide is at least 0.25-20.00, preferably at least 0.30-20.00, most preferably at least 0.50-20.00 mole negative charge per mole of monosaccharide

3. A beverage according to claim 1, wherein the added anionic polysaccharide is selected from the group consisting of iota carrageenan, kappa carrageenan, lambda carrageenan, pectin, gum Arabic, propylene glycol alginate, alginate, cellulose and starch derivatives.

4. A beverage according to claim 1, wherein the added protein has a tertiary structure.

5. A beverage according to claim 1, wherein the added protein is selected from the group consisting of caseinate, chicken egg white, bovine serum albumin and whey protein isolate.

6. A beverage according to claim 1, wherein the beverage has a pH of 2.5-6.0, preferably 3.5-5.0.

7. A beverage according to claim 1, wherein the weight ratio of added anionic polysaccharide to added protein is 20:1 to 1:4, preferably 15:1 to 1:2, most preferably 10:1 to 1:1.

8. A beverage according to claim 1, wherein the weight ratio of the combination of added anionic polysaccharide and added protein to tea solids is 0.001:1 to 1.0:1.0, preferably 0.001:1 to 0.5:1.0, most preferably 0.001:1 to 0.2:1.0.

9. A method of improving the clarity of a liquid composition comprising a polymeric polyphenol comprising either sequentially in any order or simultaneously the steps of:
a. adding a protein; and
b. adding an anionic polysaccharide.

10. A method according to claim 9, wherein step (b) is either concurrent with or precedes step (a).

11. A method according to claim 9 wherein the protein is selected from the group consisting of caseinate, chicken egg white, bovine serum albumin and whey protein isolate.

12. A method according to claim 9 wherein the anionic polysaccharide is selected from the group consisting of iota carrageenan, kappa carrageenan, lambda carrageenan, pectin, gum Arabic, propylene glycol alginate and alginate.

13. A method according to claim 9 wherein the weight ratio of anionic polysaccharide to protein is 20:1 to 1:4, preferably 15:1 to 1:2, most preferably 10:1 to 1:1.

14. A method according to claim 9 wherein the liquid composition is a pharmaceutical product or a cosmetic product or a beverage, preferably a tea-based beverage.
